(19) United States
(12) Patent Application Publication
(10) Pub. No.: US 2011/0091406 A1
(43) Pub. Date: Apr. 21, 2011

(54) COMPOSITION AND METHOD FOR PROTECTING LABILE ACTIVE COMPONENTS DURING HIGH TEMPERATURE DRYING

(76) Inventors: James V. Gruber, Washington, NJ (US); Andrew P. Banham, Jamesburg, NJ (US)

(21) Appl. No.: 12/977,326

(22) Filed: Dec. 23, 2010

Related U.S. Application Data
(63) Continuation of application No. 10/364,749, filed on Feb. 11, 2003, now abandoned.
(60) Provisional application No. 60/356,387, filed on Feb. 12, 2002.

(51) Int. Cl. A61K 8/97; A61K 8/99; A61K 8/96; A61K 36/06; A61K 35/00; A61K 35/66; A61P 17/00; A61Q 5/02; A61Q 19/10

(52) U.S. Cl. 424/74; 424/70.1; 424/195.16; 424/115

(57) ABSTRACT

A composition and method for protecting personal care components, advantageously labile active personal care components, from decomposition during high temperature drying, employing water-soluble hydrolyzed polysaccharide encapsulants. Also disclosed is an additive for a personal care composition comprising a personal care component that is at least partially encapsulated within a hydrolyzed polysaccharide encapsulant. Also disclosed is a method for protecting a composition containing labile biologically active particles which comprises encapsulating at least a portion of the biologically active particles within hydrolyzed polysaccharide particles, thereby protecting said portion of said biologically active particles.

CROSS REFERENCE TO RELATED DOCUMENT

Priority is herewith claimed under 35 U.S.C. §119(e) from copending U.S. Provisional Patent Application No. 60/356,387, filed Feb. 12, 2002, entitled “A COMPOSITION AND METHOD FOR PROTECTING LABILE ACTIVE COMPONENTS DURING HIGH TEMPERATURE DRYING,” by James V. Gruber et al. The disclosure of this U.S. Provisional Patent Application is incorporated by reference herein in its entirety.

### FIELD OF THE INVENTION

The present invention relates to a composition and method for protecting personal care components, advantageously labile active personal care components, from decomposition during high temperature drying, employing water-soluble hydrolyzed polysaccharide encapsulants.

### BACKGROUND OF THE INVENTION

The use of active components in personal care compositions continues to expand as the knowledge of human skin, its functionality and its biochemistry continues to grow. As used herein, the term “personal care compositions” is used to designate soaps, shampoos and skin care medicaments, as well as cosmetic, therapeutic, and homeopathic compositions. It is now generally accepted that the skin is not a non-living entity that simply covers the human body. Rather, it is a major organ that responds to forces from both internal biochemical signals and external physical and chemical markers. For example, even momentary contact of living skin with ultraviolet radiation, such as sunlight, causes an immediate cascade of biochemical processes designed to prevent damage from free radicals created instantly in the skin because of the radiation.

Many different eukaryotic organisms respond to external stresses in much the same way that human skin does. One example of such an organism is live yeast that is one of the simplest single-celled organisms for which the entire genome is well established. Under normal fermentation conditions yeast will grow, live and die a typical lifetime determined by their genetic make-up and environmental surroundings. Sperti recognized in U.S. Pat. No. 2,320,478 that extracts from live yeast cells comprise a cosmetic composition that will actually improve cellular respiration in cells onto which the extract is topically applied. It was further recognized in U.S. Pat. No. 2,239,345 that these yeast extracts could be further modified by applying sub-lethal stresses, such as chemicals or radiation, to the growing yeast so that the yeast responds by forming agents that are resistant to the external stress. For example, yeast exposed to ultraviolet radiation responds by forming increased amounts of cellular anti-oxidants and free radical inhibitors. The active ingredients are suitably used to provide protection to human skin when applied topically to the surface of the skin.

It is now possible to grow and maintain living human skin cultures. These cultures require very specific conditions to maintain the active growing fibroblasts, corneocytes, keratinocytes and melanocytes that comprise normal human skin. The growth medium in these cultures is typically comprised of a mixture of nutrients, vitamins and other components that the growing skin thrives on. Such “growth media” are sold commercially through, for example, PromoCell [see (http://www.promocell.com/Default.htm)] and Biowhittaker [see (http://www.medprobe.com/is/biowhittaker.html)].

Once these growth media are employed for the growth of human fibroblasts or skin substitutes, the culture media becomes “enriched” in human growth factors, polypeptides, cytokines, and other cellular components that provide the media with unique opportunities in topical applications, thus providing “enriched growth media”. Enriched growth media are also referred to herein as “conditioned media”. Disclosure regarding the use of conditioned media in topical applications is provided in PCT patent publications WO 0069449 and WO 0114527. These publications are incorporated herein by reference in their entirety.

Fermentation technology has also progressed significantly in the last decade, and it is now routinely possible for companies to bioengineer microorganisms that can produce any number of active, biologically interesting, molecules. For instance, E. coli is a commonly occurring microorganism that has been harvested to grow a number of pharmaceutically and topically active ingredients. Typically, fermentation requires the use of specialized fermentation reactors, such as those that are sold by New Brunswick Scientific [see (http://www.nbsc.com/index2.htm]. New Brunswick, N.J.). Generally, the microorganisms are grown on a nutrient broth that provides the bacteria with the essential nutrients, vitamins and other components for cell growth. Once the bacteria have grown to viability, they are typically lysed, a process that kills the bacteria, and the cellular contents are isolated as an aqueous mixture. If necessary, valuable components can be further purified if, for example, isolation of a particular pharmaceutically-active material is desired.

Active products resulting from fermentation processes have also found application in topically-applied products, as disclosed in U.S. Pat. No. 5,334,518 issued to Yakurigaku Chuo Kenkyusho. The '518 patent discloses the use of bacterial fermentation processes to manufacture gamma-pyrone derivatives for cosmetic applications.

Active products made using the above-described production methods are typically provided as aqueous or water-miscible organic solvent mixtures, such as aqueous alcohol mixtures. For example, live yeast cell derivative is typically provided as an aqueous solution that contains insoluble materials that include cell wall components. Likewise, fibroblast conditioned growth media are also typically provided as water-based compositions that contain all of the components of the fibroblast growth media plus the skin cellular components that leach from the growing fibroblasts or skin samples. Additionally, bacterial fermentation growth media typically comprise water-soluble growth factors and nutrients that are then enhanced by the presence of the bacterial lysate components.

These aqueous and aqueous alcohol active compositions (so-called “conditioned media”) have potential application as components of topical pharmaceutical, cosmetic, and personal care products. The compositions are suitable for use “as is” in water based product applications. However, in applications requiring anhydrous or substantially anhydrous components, the water-containing active compositions must be further processed to remove essentially all of the water. Drying to remove water can be effected in the presence of heat (i.e., high temperature drying) or in the absence of heat (i.e., low temperature drying).

Low temperature drying, such as freeze drying, poses certain disadvantages. For example, freeze drying is expensive because it requires freezing the components of the active composition in the presence of a vacuum to cause sublimation of the water in the composition. Another disadvantage is that many materials cannot be properly freeze dried due to the presence of salts and other low molecular weight hydroscopic materials that prevent the components from freezing correctly. More specifically, the presence of even a small amount of salt will lower the freezing point of water significantly, and can cause problems in efforts to freeze-dry a salt-containing material. Therefore, other kinds of drying need to be considered.

High-temperature drying techniques have been employed for many years in the food industry for the drying of products such as starches, vitamins and proteins for human consumption. Typical high temperature drying methods include spray drying, drum drying and flash drying. These methods typically expend significantly less energy and time, as compared to low temperature processes like freeze-drying. When using these methods of drying, the surface area of aqueous solutions or aqueous/organic solvent mixtures exposed to the drying is increased either by atomizing the solutions (as is done in spray and flash drying) or by forming films of the solutions (as is done in drum drying). The resulting increased surface area allows the products to be dried very rapidly by causing the moisture present to be exposed to the heated air. The dried products are typically collected as powders. Since the drying time is short, there is minimal contact between the active component and the hot drying surface, thus minimizing the risk of product decomposition. Nonetheless, any exposure to high temperature poses a risk of an unwanted result attributable to the drying process in view of the “labile” nature of the active component.

Labile components are unstable at elevated temperatures, and tend to undergo physical changes and/or chemical degradation at elevated temperatures. The “unwanted result” can manifest itself in various ways, such as by a color change in the product, development of an undesirable odor, or, in an extreme case, decomposition of the labile component of the composition. The latter result is particularly unacceptable since the labile components are typically the active, and hence most desirable, components of the composition, and decomposition causes loss of activity of the labile component.

Accordingly, what is needed is a new method and composition for protecting labile active compositions against heat-related physical degradation and decomposition during high temperature drying to effect water removal. The present invention provides one answer to that need.

### BRIEF SUMMARY OF THE INVENTION

It is an object of the present invention to provide a method for protecting labile active compositions against heat-related physical degradation and decomposition during high temperature drying utilized in order to effect water removal. It is another object of the present invention to provide a personal care component that is at least partially encapsulated within a hydrolyzed polysaccharide encapsulant in order to reduce or eliminate the risk of damage to the personal care component during drying at an elevated temperature. It is a further object of the present invention that the active labile components are protected from the intense heat of drying by encapsulation of the active, labile component into a hydrolyzed polysaccharide matrix. It is a further object to provide an additive suitable for use in anhydrous or essentially anhydrous personal care products. It is a further object of the present invention to provide personal care components exhibiting a “slow release” or a “timed release” characteristic due to encapsulation by means of a hydrolyzed polysaccharide matrix.

In one aspect, the present invention relates to a composition comprising an additive for a personal care composition comprising a personal care component that is at least partially encapsulated within a hydrolyzed polysaccharide encapsulant.

In another aspect, the present invention relates to a composition comprising a biologically active component encapsulated within a hydrolyzed polysaccharide matrix.

In yet another aspect, the present invention relates to a method for protecting a labile personal care component which comprises dispersing, or dissolving, the component in an aqueous or aqueous alcoholic solvent in order to provide a dispersion or solution, and drying the dispersion or solution at an elevated temperature in the presence of a hydrolyzed polysaccharide, thereby causing particles of said personal care component to become encapsulated within particles of said hydrolyzed polysaccharide.

In still another aspect, the present invention relates to a method for protecting a composition containing labile biologically active particles which comprises encapsulating at least a portion of said biologically active particles within hydrolyzed polysaccharide particles, thereby protecting said portion of said biologically active particles.

These and other aspects will become apparent upon reading the following detailed description of the invention.

### DETAILED DESCRIPTION OF THE INVENTION

It has now been surprisingly found by the present inventor that hydrolyzed polysaccharides are suitably employed in order to protect labile, active components such as live yeast cell derivatives, conditioned media or bacterial fermentation broths from the heat associated with high temperature drying of these components. Without wishing to be bound by any particular theory, it is thought that the hydrolyzed polysaccharide acts to partially dissipate the instantaneous transfer of heat into the drying particle (or “droplet”), during the drying process, by transferring the heat into the molecular structure of the hydrolyzed polysaccharide. The hydrolyzed polysaccharide thus acts as a heat sink for at least a portion of the transferred heat. This minimizes the amount of heat that the active component experiences in the drying chamber, and thus prevents undesirable decomposition reactions from occurring.

After drying, the resulting dried particle is comprised of the personal care component entrapped inside a matrix of the dry hydrolyzed polysaccharide. Because the hydrolyzed polysaccharide is essentially water soluble, it can be easily redissolved into moist environments as might be desired. Dissolution of the hydrolyzed polysaccharide will slowly release the entrained active, providing a timed-release of the active. Under certain circumstances, for example if the hydrolyzed polysaccharide is a product of starch hydrolysis, certain enzymes may also help to accelerate the dissolution of the hydrolyzed polysaccharide matrix. For instance, it is well known that the human mouth contains a variety of amylases that are known to break down the anhydroglucose bonds in starchy molecules. If the hydrolyzed polysaccharide is a starch-based material, and the composition of the present invention finds its way into a product intended for topical application to the mouth, such as, for example, lipstick, the release of the active components will be further enhanced. The use of live yeast cell derivatives in lipsticks has been suggested, for example, in U.S. Pat. No. 5,776,441, and the disclosure of this patent is incorporated herein by reference in its entirety.

As used herein, the term “hydrolyzed polysaccharide” is defined as a low molecular sugar macromonomer having a molecular weight of less that about 25,000 grams/mole, more preferably less than 10,000 grams/mole, most preferably less than 8,000 grams/mole range but greater than 1000 grams/mole. A commercially available hydrolyzed polysaccharide POLYSORB C (available from Roquette America) has a molecular weight of about 3,500 grams/mole. The hydrolyzed polysaccharides of the present invention are essentially water-soluble. “Essentially water-soluble” means that the hydrolyzed polysaccharides are soluble in water at any pH at concentrations greater than 0.5 grams per 100 grams of water at 25° C. and ambient pressure. The hydrolyzed polysaccharides useful in this invention can come from hydrolysis of a variety of polysaccharide sources including, but not limited to, hydrolyzed polyglucoses, polygalactomannans, polyglucomannans, polyarabinoses, polymannoses and the like. Especially preferred for the purposes of this invention are hydrolyzed polysaccharides derived from hydrolyzed polyglucoses such as hydrolyzed starches. Such hydrolyzed polysaccharides are available commercially from, for example, Roquette America (Koekuk, Iowa).

In the compositions of the present invention, the weight ratio of the concentration of the labile active component to the concentration of the hydrolyzed polysaccharide encapsulant suitably ranges from about 5:1 to about 1:20, preferably from about 1:1 to about 1:10, most preferably from about 1:1 to about 1:5, based upon the total weight of these two components.

As used herein, the term “anhydrous” means free of water, and the term “essentially anhydrous” means essentially free (i.e., contains less than 5 wt %) of water. “Live yeast cell derivatives” as used herein includes both aqueous and aqueous alcoholic extracts from growing yeast cultures. Such extracts may comprise, among other ingredients, vitamins, proteins, growth factors and other cellular components as disclosed, for example, in U.S. Pat. No. 2,320,478, incorporated herein by reference in its entirety. Such live yeast cell derivatives are available commercially from, for example, Arch Personal Care (South Plainfield, N.J.).

“Conditioned media” as used herein designates growth media that supports the development of human fibroblast, ketatinocytes, corneocytes or melanocytes. This media contains the nutrients, vitamins and other nutritional supplements necessary to support the growth of the skin cells along with various human growth factors, cytokines and ancillary active components excreted by the fibroblast, ketainocytes, corneocytes or melanocytes during growth. Such conditioned media are described in more detail in PCT patent publication WO PCT 01/14527 assigned to Organogenesis (Canton, Mass.) and incorporated herein by reference in its entirety.

“Bacterial fermentation media” as used herein denotes the broth that is used to support the growth of active eukaryotic or prokaryotic bacterial cultures grown aerobically or anaerobically using standard fermentation technology known to those skilled in the art. The fermentation media may comprise agar, fetal bovine serum, vitamins, minerals, and other nutritional supplements required to sustain the growth of the bacteria. In addition, nutritional supplements such as corn steep liquor, the by-product of corn wet milling, can be added to increase the nutritional content of the fermentation broth. Bacterial growth media may also include the cellular (i.e., cytoplasmic, periplasmic and nuclear) components of the bacteria, retrieved along with the growth media by lysing of the living bacteria. Such “cellular components” may include various growth factors, cytokines and polypeptides, as well as other minor components of the living cells. Cellular components are described in more detail, for example, in U.S. Pat. No. 5,334,518 and U.S. Pat. No. 6,180,367 B1, the disclosures of which are incorporated herein by reference in their entirety.

Commercial spray drying equipment useful in the present invention is available, for example, through Spray Drying Systems, Inc. (Randallstown, Md.) [see (http://www.spraydrysys.com/)]. A number of factors, principally the method of atomization, the pressure of the atomization and the temperature of the drying chamber, control particle size of the spray-dried material. Spray drying is suitably effected at a temperature from about 30° C. to about 600° C., preferably from about 400° C. to about 500° C. Typical particle sizes for spray-dried products can measure between 200 microns and 10 microns, more typically between 100 microns and 20 microns. Under these conditions, the drying process typically adversely affects each of the active components if an attempt is made to dry them without the presence of the protective hydrolyzed polysaccharide. Often this adverse effect is manifested by a color change in the product and/or development of undesirable odors attributable to the drying process.

The composition of the present invention can be used with any number of additional inert or active ingredients, as might be required to manufacture the desired therapeutic, cosmetic or personal care products. Such materials include, but are not limited to, “functional ingredients” such as, for example, conditioners, emollients, waxes, oils, polymers, fixatives, colorants, humectants, moisturizers, stabilizers, diluents, solvents, fragrances and the like, as well as “active ingredients” such as, for example, botanicals, neutraceuticals, cosmeceuticals, therapeutics, pharmaceutics, antifungals, antimicrobials, steroidal hormones, antidandruff agents, anti-acne components, sunscreens, preservatives and the like. Such additional ingredients are suitably present in an amount of from about 0.5% to about 99.9% by weight, based upon the total weight of the personal care composition.

The compositions of the present invention, being anhydrous or essentially anhydrous in nature, are suitably employed in a number of topical products and formulations. In the therapeutic, cosmetic or personal care products the composition of the present invention might be used in the range of 0.1 to 95 wt %, more preferably in the range of 0.5 to 50 wt %, most preferably in the range of 0.5 to 10 wt %. Examples of topical products into which the composition of the present invention may find use include, but are not limited to, powdered compositions such as pressed powder cosmetics, bath salts, foot powders, athletes foot treatments, anti-itch products, anti-lice products, talc and eyeshadows, shaped solid products such as lipsticks, soaps, deodorant sticks, antiperspirants, sunscreen sticks or eye pencils, solvent-based products such as nail enamels or lacquers, alcohol-based products such as sprays, body sprays, spritzes, and hair sprays, alcohol-based antimicrobial products such as lotions, sprays and towelettes, anti-comedometic products such as anti-acne products and nose strips and facial masks, oral personal care products such as toothpastes, mouthwashes, mouth deodorizers and soap compositions such as bar soaps and synthetic detergent (often called syndet) bars.

The following examples are intended to illustrate, but in no way limit, the scope of the present invention.

### EXAMPLE 1

A mixture of 500 grams of a 25 wt % aqueous solution of live yeast cell derivative available from Arch Personal Care and 1000 grams of POLYSORB C available from Roquette America, which is a 68 wt % solution of hydrogenated starch hydrolyzed polysaccharides, was prepared by adding the live yeast cell derivative to the hydrolyzed polysaccharide with vigorous mixing. Upon complete mixing of the two components the products were spray dried using a pilot scale spray dryer supplied by Niro (Soeborg, Denmark) at a temperature of about 450° C. The resulting spray dried composition was a white anhydrous powder composed of approximately 15 wt % live yeast cell derivative and 85 wt % hydrolyzed polysaccharide.

### EXAMPLES 2 AND 3

In a similar fashion to Example 1 mixtures of live yeast cell derivative at 480 and 600 grams with 520 and 400 grams of POLYSORB C hydrolyzed polysaccharide, respectively, were prepared and spray dried in a similar fashion as described above. This provided powdered compositions that comprised 20 and 35 wt % live yeast cell derivative encapsulated into 80 and 65 wt % of hydrolyzed polysaccharide, respectively.

### COMPARATIVE EXAMPLE 4

An unadulterated sample of 25 wt % live yeast cell derivative (but containing no hydrolyzed polysaccharide) was spray dried using similar conditions as described above in Example 1. The resulting dry powdered product was discolored to a deep brown and had an offensive, burnt odor.

### EXAMPLE 5

A sample of 200 grams conditioned growth medium was blended with 200 grams of POLYSORB C hydrolyzed polysaccharide. The mixture was spray dried using conditions similar to those described in Example 1. The resulting white powder comprised approximately 30 wt % of conditioned growth media encapsulated in approximately 70 wt % hydrolyzed polysaccharide.

While the invention has been described above with reference to specific embodiments thereof, it is apparent that many changes, modifications, and variations can be made without departing from the inventive concept disclosed herein. Accordingly, it is intended to embrace all such changes, modifications and variations that fall within the spirit and broad scope of the appended claims. All patent applications, patents and other publications cited herein are incorporated by reference in their entirety.

1-14. (canceled)

15. An additive for a personal care composition selected from the group consisting of soaps, shampoos, skin care medicaments, and cosmetics comprising
a personal care component comprising a labile active material selected from the group consisting of live yeast cell derivatives, human fibroblast conditioned media, plant and bacterial fermentation products, and combinations thereof,
wherein the labile active material is fully encapsulated within a hydrolyzed polysaccharide encapsulant in order to protect the labile active material against physical or chemical degradation attributable to exposure to an elevated temperature of from about 400° C. to about 500° C.,
wherein the hydrolyzed polysaccharide is a low molecular weight sugar macromonomer having a molecular weight of from 1,000 grams/mole to 8,000 grams/mole, and
wherein the weight ratio of the labile active material to the hydrolyzed polysaccharide ranges from about 1:1 to about 1:5.

16. The additive of claim 15 wherein the hydrolyzed polysaccharide is a hydrolyzed starch.

17. The additive of claim 15 wherein the hydrolyzed polysaccharide is hydrogenated starch hydrolyzed polysaccharides.

18. The additive of claim 15 wherein the labile active material is consisting of live yeast cell derivatives.

19. The additive of claim 15 wherein the labile active material is an anhydrous powder.

20. A topical composition comprising from 0.5 to 10% of a labile active material selected from the group consisting of live yeast cell derivatives, human fibroblast conditioned media, plant and bacterial fermentation products, and combinations thereof,
wherein the labile active material is fully encapsulated within a hydrolyzed polysaccharide encapsulant in order to protect the labile active material against physical or chemical degradation attributable to exposure to an elevated temperature of from about 400° C. to about 500° C.,
wherein the hydrolyzed polysaccharide is a low molecular weight sugar macromonomer having a molecular weight of from 1,000 grams/mole to 8,000 grams/mole, and
wherein the weight ratio of the labile active material to the hydrolyzed polysaccharide ranges from about 1:1 to about 1:5.

21. The topical composition of claim 20 wherein the hydrolyzed polysaccharide is a hydrolyzed starch.

22. The topical composition of claim 20 wherein the hydrolyzed polysaccharide is hydrogenated starch hydrolyzed polysaccharides.

23. The topical composition of claim 20 wherein the labile active material is consisting of live yeast cell derivatives.

24. A method for protecting a labile personal care component which comprises dispersing or dissolving the component in an aqueous or aqueous alcoholic solvent in order to provide a dispersion or solution, drying the dispersion or solution at from 400° C. to about 500° C. in the presence of a hydrolyzed polysaccharide, thereby causing the particles of said personal care component to become fully encapsulated within particles of said hydrolyzed polysaccharide,
wherein labile personal care component is selected from the group consisting of live yeast cell derivatives, human fibroblast conditioned media, plant and bacterial fermentation products, and combinations thereof,
wherein the hydrolyzed polysaccharide is a low molecular weight sugar macromonomer having a molecular weight of from 1,000 grams/mole to 8,000 grams/mole, and
wherein the weight ratio of the labile personal care component to the hydrolyzed polysaccharide ranges from about 1:1 to about 1:5.

25. The method of claim 24 wherein the hydrolyzed polysaccharide is a hydrolyzed starch.

26. The additive of claim 24 wherein the hydrolyzed polysaccharide is hydrogenated starch hydrolyzed polysaccharides.

27. The additive of claim 24 wherein the labile active material is consisting of live yeast cell derivatives.
